(19) United States
(12) Patent Application Publication
(10) Pub. No.: US 2019/0209760 A1
(43) Pub. Date: Jul. 11, 2019

(54) SYSTEM FOR EXTRACORPOREAL MEMBRANE OXYGENATION WITH A BLOOD PUMP AND AN OXYGENATOR

(71) Applicant: Xenios AG, Heilbronn (DE)

(72) Inventor: Ivo SIMUNDIC, Wendlingen (DE)

(73) Assignee: Xenios AG, Heilbronn (DE)

(21) Appl. No.: 16/302,151

(22) PCT Filed: Apr. 27, 2017
(86) PCT No.: PCT/DE2017/000116

§ 371 Date: Nov. 16, 2018

(30) Foreign Application Priority Data
May 18, 2016 (DE) 10 2016 006 013.1

(51) Int. Cl. A61M 1/16; A61M 1/10; A61M 1/36

(52) U.S. Cl. CPC A61M 1/1631; A61M 1/1086; A61M 1/1698; A61M 1/3641; A61M 2230/04; A61M 1/3626; A61M 2205/3334; A61M 2205/3365; A61M 2206/22; A61M 1/1036

(57) ABSTRACT

In a system for extracorporeal membrane oxygenation including a blood pump and an oxygenator, the oxygenator includes fibrous mats stacked in a housing and arranged parallel to one another, and the blood pump includes a control unit that provides for a continuous variation of the volume of flow over time.

The invention relates to a system for extracorporeal membrane oxygenation with a blood pump and an oxygenator, wherein the blood pump comprises a control which allows continuous variation of the flow volume over time.

Continuous variation of the flow volume over time is taken to mean a variation of the flow volume which is not due to the switching on and off of the blood pump. It is also not taken to mean changes in the flow volume due to the type of blood pump, such as, for example, in the case of a roller pump or also a centrifugal pump which due to their design do not generate an absolutely continuous flow.

The continuous variation of the flow volume over time results from control-related overlapping of the control of the pump through a variation of the flow volume as a result of an external influence on the control of the pump. Through this, a pulsatile flow, for example, can come about which like a sinus curve or a rectangular impulse oscillating over a longer time brings about a variation in the flow volume. However, on the basis of measurements matched to the current situation of the patient's body, volumetric flow changes can be made which in turn, preferably cyclically matched to the heartbeat, determine a defined volumetric flow rate or a defined variation of the volumetric flow.

Through the variation in the flow volume the operation of the system for extracorporeal membrane oxygenation can be adapted to the patient's current heart situation. Doctor-determined deviations from a continuous volumetric flow, particularly in terms of a change in amplitude and/or wavelength, can be implemented and a pulsatile control of the blood pump can also be used to loosen air bubbles stuck in the system in order to vent the system more quickly.

Such a system is known from DE 10 2013 012 433 A1 for example.

For these systems a device as described in EP 0 765 689 is used as the oxygenator. This makes it possible to enrich the blood with oxygen and bring it to the correct temperature in a simple manner. These oxygenators also cushion pressure peaks occurring on the blood pump.

A system of this type is generally connected via cannulas to a person's blood circulation, in particular to the heart. The pulsatility at the outlet on a cannula arranged in the heart should correspond to a pulsatility precisely predetermined by the doctor. As it would be laborious to measure the pulsatility at the outlet of the cannula in the heart, the flow values of the blood are measured at the pump outlet and in relation to the system of blood pump, oxygenator and cannulas the doctor estimates when setting the pump control what pulsatility is to be expected at the outlet of the cannula in the heart.

The aim of the invention is to develop such a system further.

According to a first aspect according to the invention the oxygenator of the system comprises mats with fibres stacked in a housing which are arranged in parallel to each other.

Blood can flow through a membrane oxygenator of this type perpendicularly to the stacked mats and then exhibits a particularly small drop in pressure. This results in a value relating to the flow behaviour of the blood measured at the oxygenator that largely corresponds to the value at the outlet of the cannula in the heart.

As the pulsatile blood flow through the oxygenator hardly changes, the pulsatility before the oxygenator corresponds almost exactly to the pulsatility after the oxygenator. This makes for a pulsatile blood flow at the system outlet and at the same time gentle acceleration of the blood at the pump within the system.

Particularly advantageous is a form of embodiment in which the control is connected to an ECG. This makes it possible for the values measured with the ECG to be used directly or in a processed form for controlling the blood pump. Through this, in the simplest case the cycle measured by the ECG can be used for controlling the pump. In addition, offsetting the ECG values enables systematic control to take place in order to determine the flow behaviour at the heart via the blood pump.

A further aspect of the invention relates to the design of the rotor. Particularly advantageous has proven to be a blood pump which has a rotor, the outer diameter of which is smaller than 4 cm, preferably smaller than 3.5 cm, and has diameter of more than 1 cm. Such a small rotor results in a small pump housing. In addition, by reducing the rotor diameter its moment of inertia decreases. This makes it possible to change the rotor speed particularly quickly in order to achieve a special flow profile of the volumetric flow over time. A larger rotor diameter could build up a greater pressure in order to overcome the resistance. However, such large rotors are necessarily sluggish and imprecise. Through the special moment of inertia the proposed rotor diameters make for optimum timing for the pulse. This is advantageous for the use of any type oxygenator. Particularly in connection with mats with fibres, arranged in parallel to each other stacked in a housing, this flow profile is maintained largely unchanged when the blood flows through the system up the cannula outlet in the heart.

It is therefore proposed that the blood pump comprises a rotor, the moment of inertia is less than 5000 g/mm² and preferably less than 1000 g/mm². In order to guarantee the functioning of the blood pump, its moment of inertia is greater than 200 g/mm². Positive results were achieved with a rotor with a moment of inertia is around 660 g/mm².

It is advantageous if the blood pump has a rotor which is connected via magnets to an actuator which rotates the rotor about an axis, wherein the magnets are arranged at an average distance of 5 to 10 mm from the axis. On the one hand this results in optimised functioning of the magnets which should only increase the moment of inertia of the rotor slightly, and on the other hand allows good coupling between the rotor and motor via the magnets.

Suitable above all as blood pumps are blood pumps which only have a small radial flow portion. It is therefore proposed that the blood pump has a rotor which results in an axial flow portion. In particular these are axial or diagonal pumps.

A particularly advantageous design of the oxygenator envisages that the cross-section of the blood inflow before the mat is increased in order to reduce the flow speed of the blood. On the way to the oxygenator this makes it possible to convey blood to the oxygenator with a small line diameter and high flow speeds and shortly before the mat into which the blood flows to reduce the speed of the blood in that there the cross-section that is available for the flow is considerably increased.

Accordingly the cross-section of the blood inflow can also be reduced after the flowed-through mat in order to increase the flow speed of the blood.

A special form of embodiment representing a further aspect of the invention envisages that a pressure relief device is arranged between the pump and oxygenator. In this way pressure peaks can be intercepted in order to reduce the mechanical loading of the pump and/or oxygenator. The pressure relief device is preferably arranged in the direction of flow between the pump and the oxygenator. An equalisation vessel with an air cushion or a line made of flexible material can be used a pressure relief device. Such a flexible line can be briefly dilated in order to intercept a pressure peak by increasing the cross-section.

However, pressure relief can also be achieved through a flexible or flexibly borne blood distribution plate. Such a blood distribution plate has the additional advantage that direct flow to the hollow fibres is prevented.

It is also proposed that the pressure relief device comprises an equalisation vessel with a gas cushion or a line with at least one flexible wall area.

Another possibility of cushioning pressure surges is the movable holding of the mats in the oxygenator in a frame. In particular, if the mats of the oxygenator are held movably in a frame, it is advantageous if the frame is held movable relative to the housing. Through this the mats are ultimately movable relative to the housing as a result of which cushioning of a brief pressure peak can be achieved.

More particularly, the combination of variation of the flow volumetric flow over time with, if necessary, only slight cushioning of the volumetric flow peaks, leads to an effective throughflow that protects the mats.

A compact structure and protective handling of the blood is also achieved in that a connection line between the blood pump and the oxygenator is less than 20 cm, preferably less than 15 cm and particularly preferably less than 5 cm in length. The blood pump and the oxygenator are thus arranged as close as possible to each other and a preferably even held in the same housing.

Other advantages come about through different embodiments of the mats and in particular through the arrangement of the mats relative to each other. Thus, it is initially envisaged that the mats are arranged at an angle of 90° to each other from one to the next plane. The angle describes the angle between main flow directions of mats borne on top of each other.

It is also proposed that the mats are rectangular and preferably quadratic in design.

A further form of embodiment envisages that the oxygenator comprises a cylindrical housing in which the mats are arranged in parallel to an orthogonal sectional circular area of the cylindrical housing.

A simple structure is produced in that the oxygenator has a central inlet and preferably also a central outlet. Alternatively the oxygenator can also have a decentral inlet and a decentral outlet.

A particularly preferred embodiment variant envisages that the oxygenator has an air bubble sensor. This makes it possible to determine gas present in the system directly at the oxygenator in order, if necessary, to expel the gas out of the oxygenator through a variation in the flow volume, for example through switching to pulsatile inflow.

Advantageous embodiment variants are produced depending on the purpose of use, in that the oxygenator is downstream of the pump or the pump is downstream of the oxygenator.

Advantageous forms of embodiment are shown in the drawing and will be explained in more detail below. Here

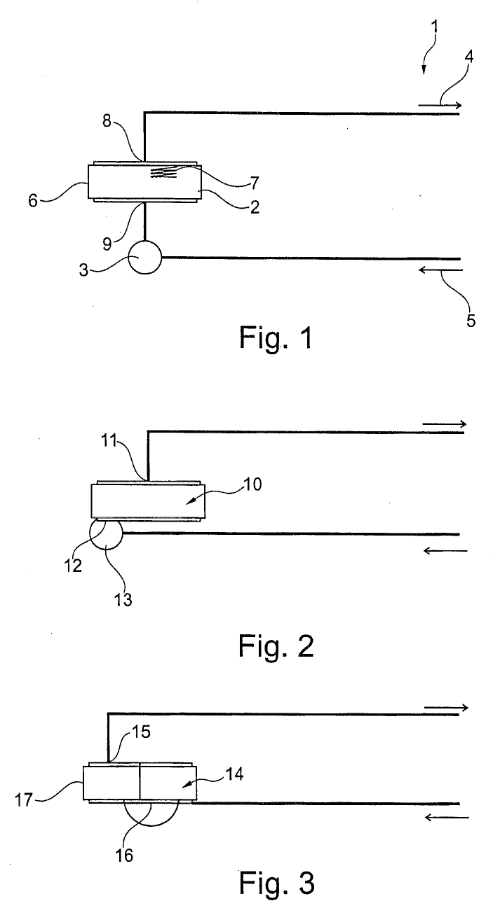

FIG. 1 shows a round oxygenator with a central connection for the inlet and outlet,

FIG. 2 schematically shows a round oxygenator with a decentral inlet and outlet,

FIG. 3 schematically shows an angular oxygenator with a decentral inlet and outlet,

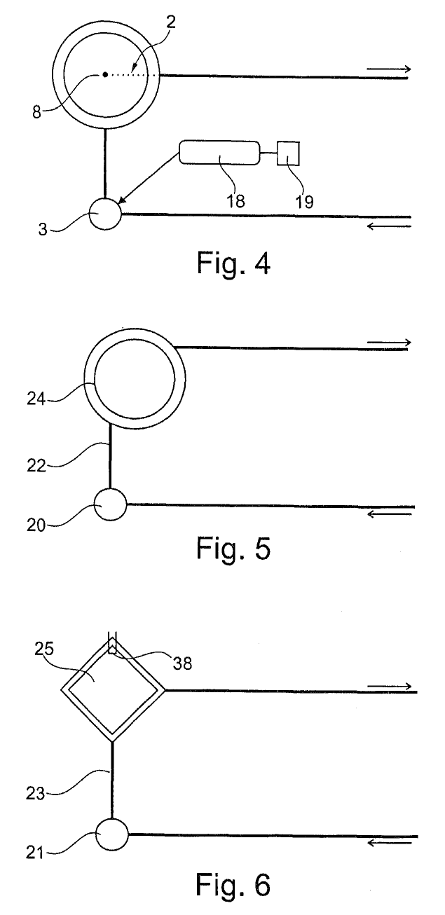

FIG. 4 schematically shows a round oxygenator with a central connection and a control,

FIG. 5 schematically shows a round oxygenator with a decentral connection,

FIG. 6 schematically shows an angular oxygenator with a central connection,

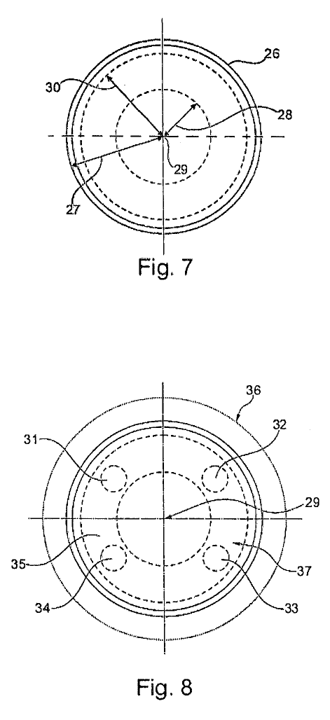

FIG. 7 schematically shows a section in sectional plane orthogonal to the rotor axis of the pump under the rotor of the pump without magnets,

FIG. 8 schematically shows the section shown in FIG. 7 with magnets and

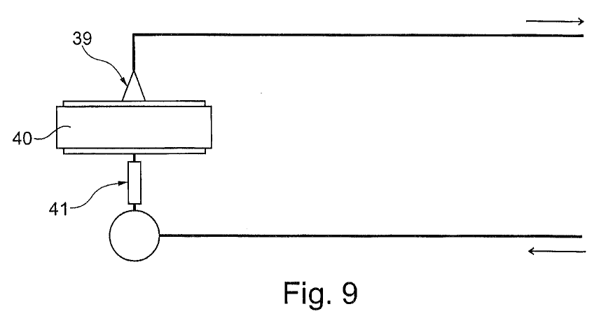

FIG. 9 schematically shows a round oxygenator with a central connection with a blood inlet increasing in cross-section and equalisation vessel.

The system 1 shown in FIG. 1 comprises a round oxygenator 2 and a pump 3. The direction of flow, indicated by the arrows 4 and 5, shows that the flow initially passes through the pump 3 and then the oxygenator 2. The oxygenator 2 has a housing 6 in which schematically indicated mats 7 which have hollow fibres are stacked.

As a first connection the oxygenator 2 has a central inlet 9 and as a second connection a central outlet 8.

FIG. 2 shows a similar configuration in which the oxygenator 10 has a decentral inlet 12 which is directly connected to the pump 13, and a decentral outlet 11.

FIG. 3 shows a further oxygenator 14 which comprises a central inlet 16 and a decentral outlet 15 and the housing 17 of which is quadratic in design.

FIG. 4 shows a view from above of the round oxygenator 2 shown in FIG. 1 with the covered central connection 8 and the blood pump 3 which is in connection with a control 18 which makes continuous variation of the flow volume over time possible. The control 18 is in connection with the schematically indicated ECG 19.

FIGS. 5 and 6 respectively shows a view of the systems according to FIGS. 2 and 3 in a schematically similar configuration—but with a blood pump 20 and 21 respectively which is connected by means of 22 and 23 respectively to the cylindrical oxygenator 24 and the quadratic oxygenator 25 respectively.

FIG. 7 shows the underside of a rotor 26 which has an external diameter 27 of around 2.5 or 3 cm. The smaller mean radial distance of the magnets from the axis 29 of 5 mm is indicated with the arrow 28 and the larger mean radial distance of 10 mm is indicated with the arrow 30.

Several magnets 31, 32, 33 and 34 are arranged as small round pieces of magnet in the ring 35. Either 4 to 8 small magnet pieces or a ring magnet can be used as magnets. These are arranged concentrically about the axis 29 within a housing 36 and form a magnet system 37 with which the force of an actuator (not shown) is transferred to the rotor 26.

In FIG. 6 an oxygenator 25 with an air bubble sensor 38 is schematically indicated.

FIG. 9 shows an outlet 39 from the oxygenator 40 which widens in a funnel-shaped manner shortly after the oxygenator so that the blood flows to the mats in the oxygenator at a slower rate than in the line after the oxygenator. Accordingly, in the direction of flow an inlet can be provided before the oxygenator which widens in a funnel-like manner in order to protect the plates to which the flow is directed. Such a funnel-like inlet or outlet is suitable for any type of oxygenator in order to reduce the flow speed in the oxygenator and thereby protect the plates to which the flow is directed.

FIG. 9 also shows an equalisation vessel 41 in which a gas cushion, is provided in order to cushion a fluctuating pressure in the system. Such an equalisation vessel 41 can be arranged at any point of the system and preferably in the vicinity of the oxygenator.

The figures show a flow through the system in which the blood first flows through the blood pump and then the oxygenator. However, the flow can also pass through the system in the opposite direction so that the blood first flows through the oxygenator and thereafter the blood pump.

1. A system for extracorporeal membrane oxygenation (1) with a blood pump (3, 13) and an oxygenator (2, 10), wherein the blood pump (3, 13) comprises a control (18) which makes continuous variation of the flow volume over time possible, wherein the oxygenator (2, 10) comprises mats (7) with fibers stacked in a housing (6) which are arranged in parallel to each other.

2. The system according to claim 1, wherein the control (18) is in connection with an ECG (19).

3. The system according to claim 1, wherein the blood pump (3, 13) comprises a rotor (26), the outer diameter (27) of which is smaller than 4 cm, preferably smaller than 3.5 cm.

4. The system according to claim 1, wherein the blood pump (3, 13) comprises a rotor (26) the moment of inertia of which is less than 5000 g/mm² and is preferably smaller than 1000 g/mm².

5. The system according to claim 1, wherein the blood pump (3, 13) comprises a rotor (26) which by way of magnets (31 to 34) is in connection with an actuator which rotates the rotor (26) about an axis (29), wherein the magnets (31 to 34) are arranged at a means radial distance (28, 30) of 5 to 10 mm from the axis (29).

6. The system according to claim 1, wherein the blood pump (3, 13) comprises a rotor (26) which brings about an axial flow portion.

7. The system according to claim 1, wherein a pressure relief device is arranged between the blood pump (3, 13) and oxygenator (2, 10).

8. The system according to claim 7, wherein the pressure relief device comprises an equalization vessel (41) with a gas cushion.

9. The system according to claim 7, wherein pressure relief device comprises a line with at least one flexible wall area.

10. The system according to claim 1, wherein the connection line (22, 23) between the blood pump (3, 13) and the oxygenator (2, 10) is less than 20 cm, preferably less than 15 cm and particularly preferably less than 5 cm in length.

11. The system according to claim 1, wherein the cross-section of the blood inlet (39) before the mat to which the flow is directed is enlarged in order to reduce the flow speed of the blood.

12. The system according to claim 1, wherein the mats of the oxygenator are held in movable manner in a frame.

13. The system according to claim 12, wherein the frame is held in a movable manner relative to the housing.

14. The system according to claim 1, wherein the mats arranged at an angle of 90° from one to the next plane.

15. The system according to claim 1, wherein the mats are rectangular and preferably quadratic in design.

16. The system according to claim 1, wherein the oxygenator (2, 10) has a cylindrical housing in which the mats are arranged in parallel to a sectional circular area.

17. The system according to claim 1, wherein the oxygenator (10) has a decentral inlet (12) and preferably also a decentral outlet (11).

18. The system according to claim 1, wherein the oxygenator (2) has a central inlet (9) and preferably also a central outlet (8).

19. The system according claim 1, wherein oxygenator (25) has an air bubble sensor (38).

20. The system according to claim 1, wherein the oxygenator (3, 13) is downstream of the blood pump (3, 13).

21. The system according to claim 1, wherein the blood pump is downstream of the oxygenator.
